(19) United States
(12) Patent Application Publication
(10) Pub. No.: US 2023/0017546 A1
(43) Pub. Date: Jan. 19, 2023

(54) METHODS AND SYSTEMS FOR REAL-TIME CYCLE LENGTH DETERMINATION IN ELECTROCARDIOGRAM SIGNALS

(71) Applicant: GE Precision Healthcare LLC, Milwaukee, WI (US)

(72) Inventors: Dan R. Schneidewend, Menomonee Falls, WI (US); Sean Blachley, Milwaukee, WI (US); Steve Schulz, Rubicon, WI (US); Joel Qiuzhen Xue, Wauwatosa, WI (US)

(21) Appl. No.: 17/379,812

(22) Filed: Jul. 19, 2021

(51) Int. Cl. A61B 5/35; A61B 5/00; A61B 5/283

(52) U.S. Cl. CPC A61B 5/35; A61B 5/7246; A61B 5/7225; A61B 5/283; A61B 5/7264; A61B 5/743

(57) ABSTRACT

Various methods and systems are provided for analyzing an electrocardiogram (ECG) in real-time using machine learning to identify heartbeats, calculate a cycle length for each heartbeat, and display the cycle length for each heartbeat at a user interface. Waveform morphology of ECG data is continuously learned to identify recurrent signals and generate templates based on recurrent signals, to which ECG data is compared to identify and display heartbeats. Generated templates are continuously updated to reflect changing waveform morphologies.

### FIELD

Embodiments of the subject matter disclosed herein relate to the analysis of electrocardiograms (ECGs), and more specifically, to methods and systems for real-time cycle length determination using machine learning to identify individual cardiac activations and measure activation timing.

### BACKGROUND

Heart disease has become the most common disease that affects humans worldwide. Each year millions of people die from heart attacks and an equal number undergo coronary artery bypass surgery or balloon angioplasty for advanced heart disease. Early detection and timely treatment may reduce the probability and/or severity of such events. Early detection may improve the quality of life and slow the progression of heart failure.

Both invasive and non-invasive approaches to collect electrocardiograms (ECGs) may be used to assess a patient's heart condition, which may enable early detection of cardiac irregularities. An ECG may utilize surface electrodes, e.g., electrodes in contact with the patient's skin, and/or an ICEG (intracardiac electrogram), e.g., insertion of one or more electrodes via catheters into chambers of the heart to monitor cardiac activity and may each offer distinct advantages. When used in tandem, the combination of external and internal monitoring may enable a relatively accurate and fast diagnosis. Both methods may generate electrocardiograms to provide information on the normal and/or pathological physiology of the heart and may be used to diagnose one or more cardiac conditions, such as arrhythmias, cardiac infarctions, cardiac hypertrophy, etc.

An overall magnitude and direction of the heart's electrical depolarization is captured by an electrocardiogram at each moment throughout the cardiac cycle, or the alternating contraction and relaxation of the heart chambers. Each cardiac cycle, or heartbeat, may have a cycle length of approximately 0.8 seconds in a healthy adult. For individuals with heart disease, such as arrhythmia, a cycle length may be shorter or longer compared to that of a healthy heart due to irregular heartbeats. Estimating a cycle length of each heartbeat by identifying signals of ECG data, where a signal is a peak of electrical potential signifying a heartbeat, and calculating a length of time therebetween, assists physicians in diagnosing arrhythmia by identifying regions of the heart with varying signal to signal cycle lengths, indicating irregular heartbeats.

### BRIEF DESCRIPTION

In one embodiment, a method includes establishing a plurality of standardized electrocardiogram (ECG) patterns based on real-time sensed ECG data and selecting signals of the real-time sensed ECG data based on a correlation between the real-time sensed ECG data and at least one pattern of the plurality of standardized ECG patterns. Furthermore, a cycle length of each of the signals of the real-time ECG data is determined and the cycle length for the respective signal is displayed at a user interface. The method is executed continuously, in parallel, and independently at each channel of an ECG and ICEG system. In this way, continuous learning of waveform morphology is enabled, thereby allowing accurate identification of dynamic ECG signals in real-time.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 shows a block diagram of an exemplary embodiment of an ECG system.

FIG. 2 shows a flow chart for an example of a high-level method for calculating a cycle length based on ECG data.

FIG. 3 shows a flow chart for an example of a method for calculating the cycle length from ECG data in conjunction with the method of FIG. 2.

FIGS. 4A-4C show example ECG data segments utilized by the methods of FIGS. 2 and 3 to calculate the cycle length.

FIG. 5 shows a flow chart for an example of a method for calculating the cycle length from ECG data in conjunction with the methods of FIGS. 2 and 3.

FIGS. 6A-6B show example ECG data segments utilized by the methods of FIGS. 2 and 5 to calculate the cycle length.

FIG. 7 shows an example ECG display.

FIG. 8 shows example ECG displays including graphs of a healthy heart and a heart with arrhythmia.

### DETAILED DESCRIPTION

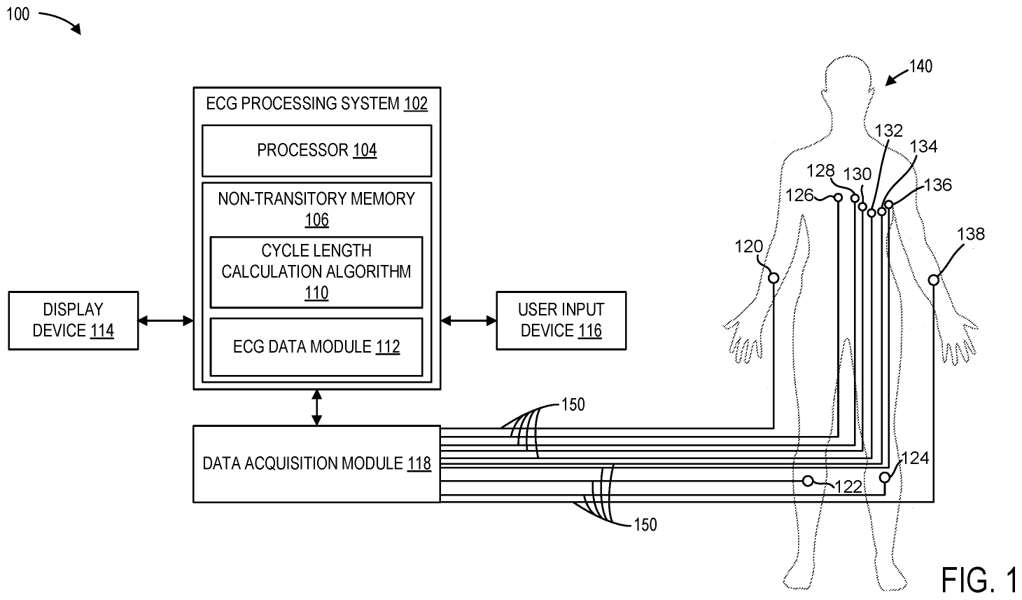

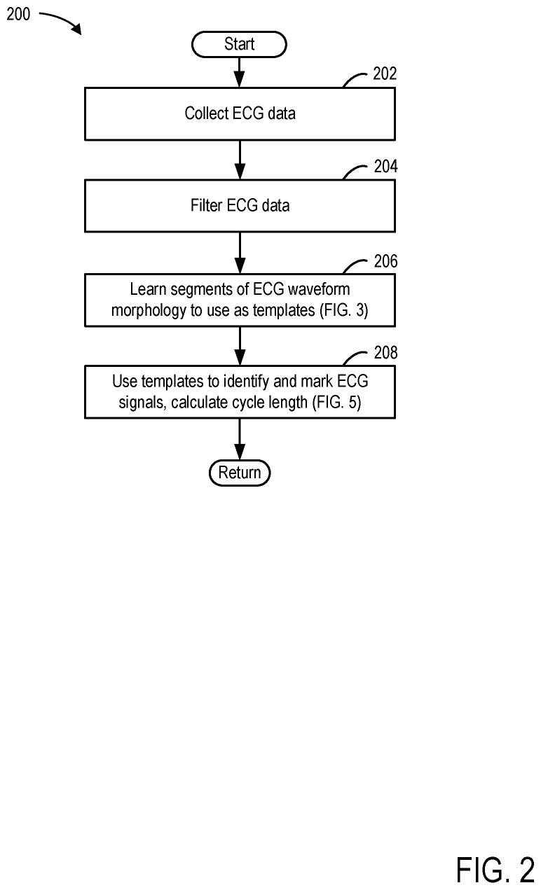

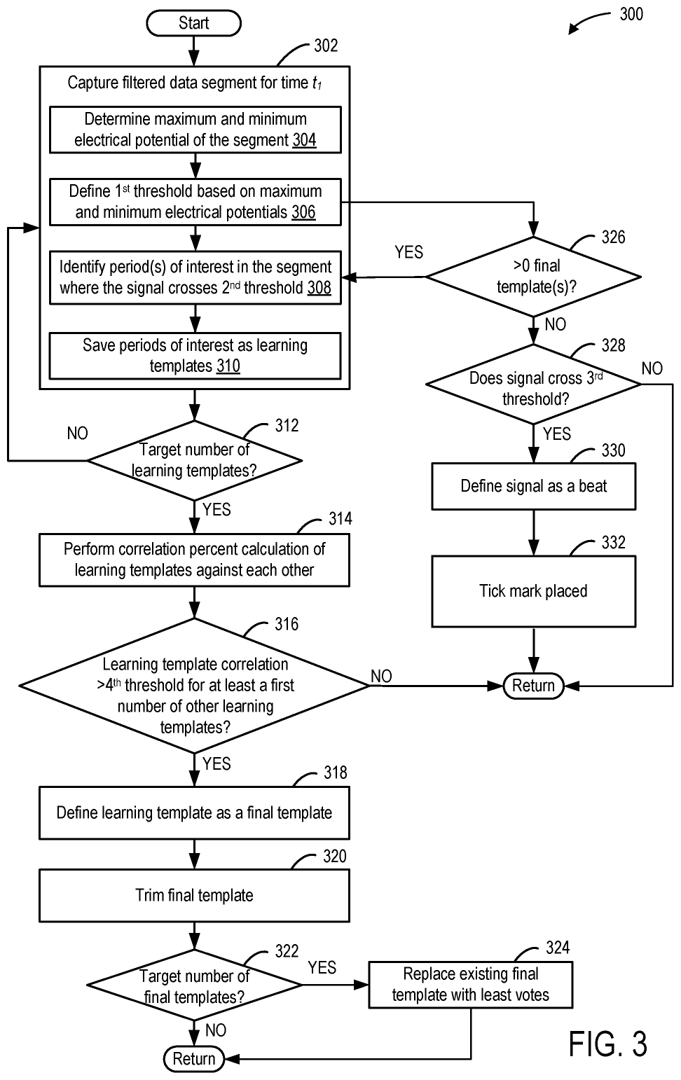

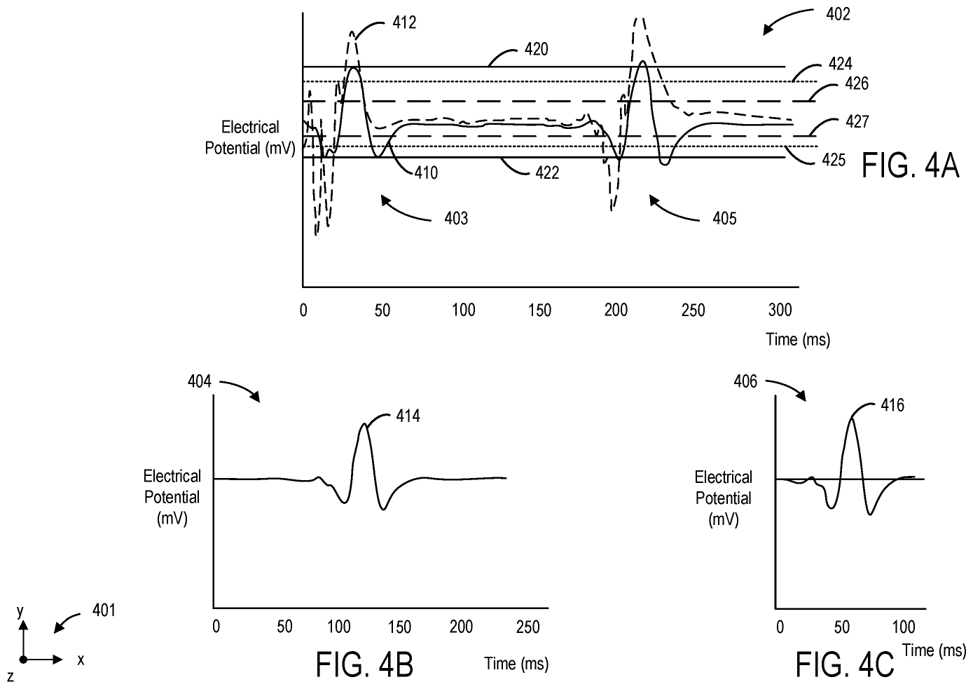

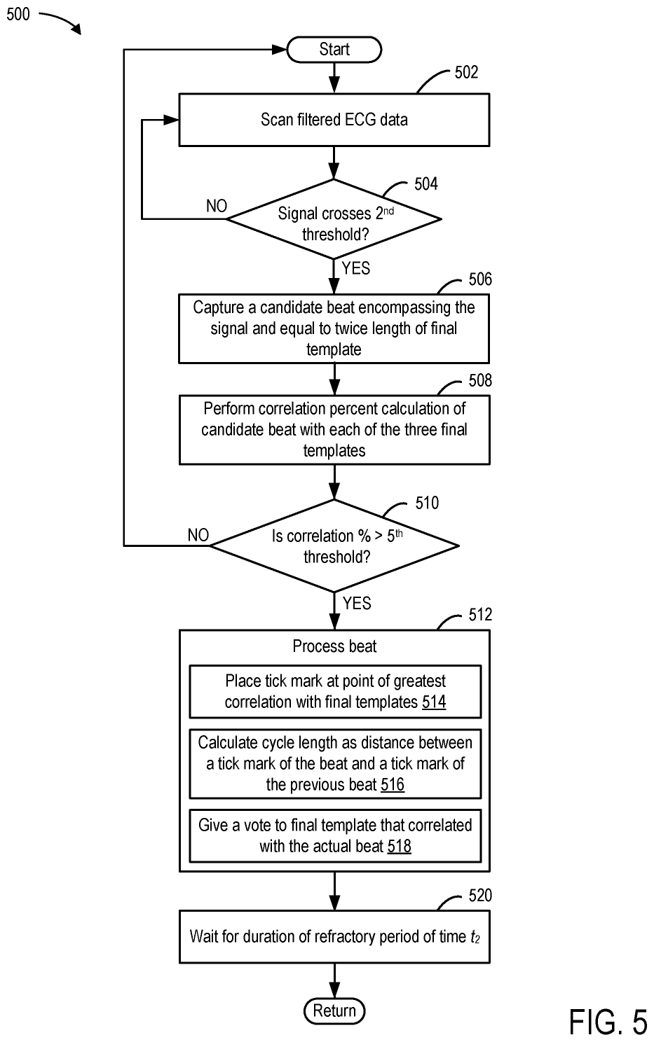

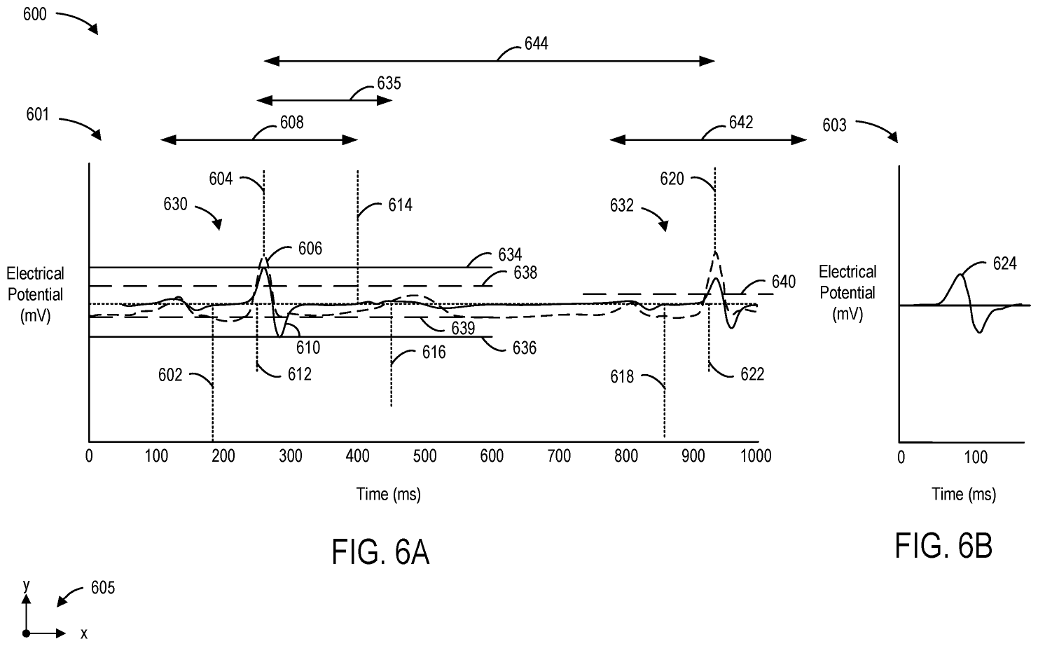

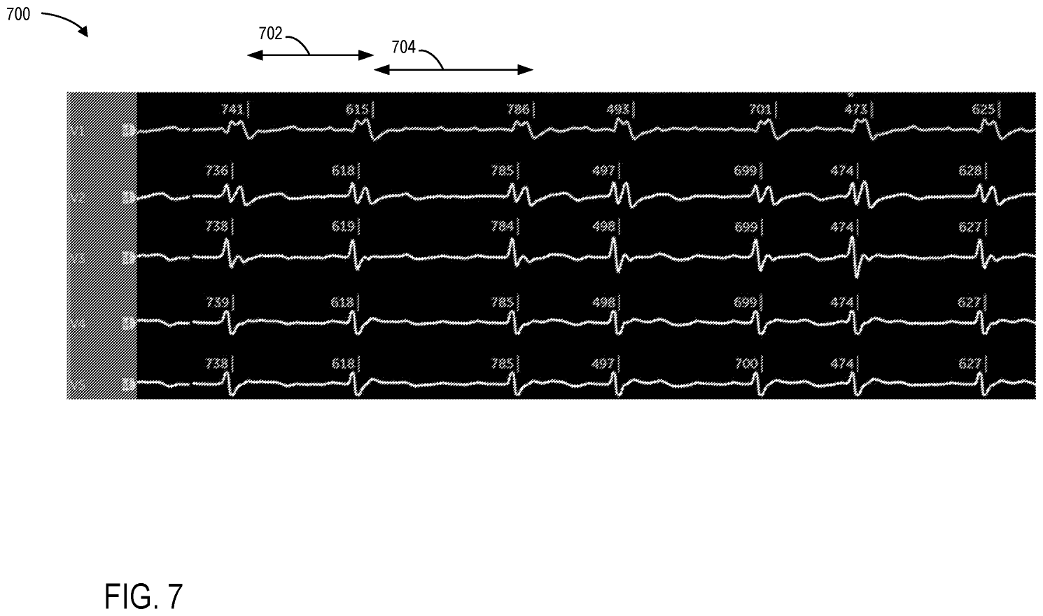

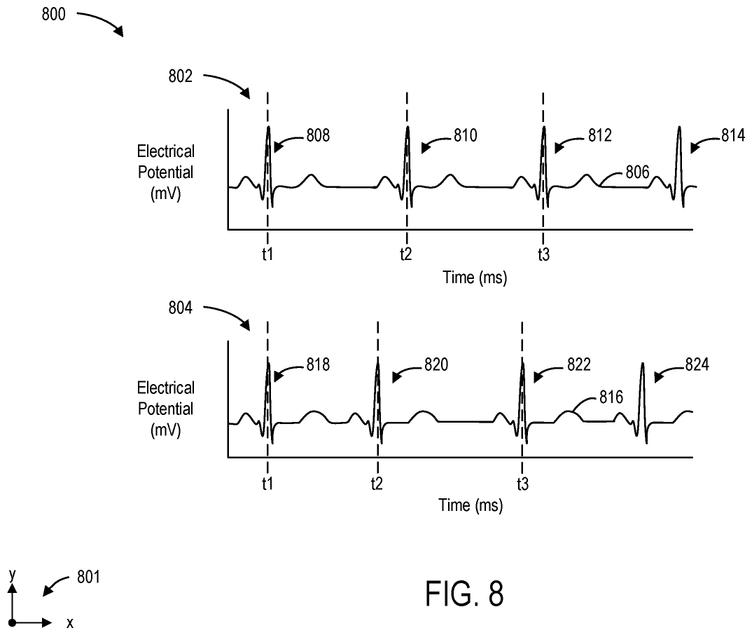

The following description relates to systems and methods for real-time cycle length determination using machine learning to identify individual cardiac activations. Real-time determination refers to collecting, filtering, and analyzing electrocardiogram (ECG) data while the ECG is being performed on a patient. Machine learning may include, but is not limited to, training one or more algorithms using statistical methods to make classifications and/or predictions based on a set of data. Example of methods for training the one or more algorithms are depicted in FIGS. 2, 3, and 5. Cardiac activation, herein referred to as “heartbeat”, refers to electrical depolarization and repolarization of a heart. A cycle length is a duration of the heartbeat, or the time it takes for a heart to electrically depolarize and repolarize, and is calculated as a time between heartbeats. An example ECG system used to collect ECG data for determining cycle length is shown in FIG. 1. The ECG system may be a surface ECG, where electrodes are placed on a patient's skin, an intracardiac electrogram (ICEG), where electrodes are disposed on a catheter which is inserted into the patient's heart chambers, or a combination of the ECG and ICEG. FIG. 8 shows example ECG displays including graphs of ECG data for a healthy heart and for a heart experiencing arrhythmia, as may be collected by the ECG system of FIG. 1. Data collected by the ECG system of FIG. 1 is processed according to a method for calculating a cycle length of a recurrent signal of the ECG data, as shown in FIG. 2, where a recurrent signal of ECG data refers to a signal with the same waveform morphology as multiple other signals, as further described in FIG. 8. FIG. 3 elaborates on the method of FIG. 2 and describes a method for generating final templates from the ECG data. FIGS. 4A-C show example graphs of a segment of ECG data from which a final template is generated, an example learning template, and an example final template, respectively. A final template generated according to the method of FIG. 3, and an example of which is shown in FIG. 4C, is used to identify and mark recurrent patterns or signals in the ECG data, as described by the method of FIG. 5. FIG. 6A shows an example graph of a segment of ECG data to which a final template, as shown in FIG. 6B, may be matched to identify a recurrent signal in the segment. FIG. 7 shows an example display, such as a display of the ECG system of FIG. 1, where recurrent signals are marked and cycle lengths are labeled.

Referring to FIG. 1, an electrocardiogram (ECG) system 100 is shown, in accordance with an exemplary embodiment. ECG system 100 comprises a signal sensing unit, which may take different forms, such as one or more ICEG intracardiac leads, a standard 12-lead ECG, or a combination thereof. An intracardiac lead may be configured as a conventional catheter having multiple electrodes supported along a distal portion of the catheter. The catheter may be disposed within or moved around chambers of the heart to collect intracardiac ECG data. In the illustrated embodiment, the signal sensing unit is represented as a standard 12-lead ECG for brevity, however one or more catheters may be included or used in place of or in tandem with the 12-lead ECG. The catheter may be electrically coupled to the ECG system 100 in a similar fashion as described below with reference to the 12-lead ECG.

The 12-lead ECG, shown in FIG. 1, may include a set of electrodes 150, attached to a patient 140. Electrodes 150 are electrically coupled to a data acquisition module 118, thus enabling data acquisition module 118 to measure ECG waveform data by determining a difference in electrical potential between two or more electrodes of electrodes 150. Data acquisition module 118 is communicatively coupled to ECG processing system 102, for processing, storing, and/or diagnosing, ECG data acquired by data acquisition module 118. ECG processing system 102 is further communicatively coupled to a display device 114, which is configured to display ECG data and/or diagnoses determined for the ECG data and may be part of a user interface, as well as user input device 116, which may enable a user to enter data into ECG processing system 102 or interact with data within non-transitory memory 106. Further, ECG system 100 may be communicatively coupled to one or more client devices (not shown), such as through a network connection, or through the Internet.

Data acquisition module 118 is configured to acquire time traces of electrical potentials between two or more of electrodes 150, which may include using an amplifier (not shown) to amplify ECG signals. In ECG system 100, electrodes 150 include ten electrodes, configured to measure a twelve lead ECG. In some embodiments, ECG system 100 may include 13 electrodes and may be configured to acquire fifteen lead ECG data. The methods and systems described herein apply to any multiple lead ECG system, including, but not limited to, a twelve lead ECG system.

Although the meaning of a twelve lead ECG will be well understood by a person having ordinary skill in the art, briefly, a twelve lead ECG provides a record of the electrical potential of the heart, as a function of time, measured along twelve distinct axes intersecting the heart. Therefore, a twelve lead ECG includes twelve distinct, time varying signals, wherein each of the twelve distinct time varying signals represents the electrical activity of the heart measured along a different axis. The electrodes 150 may include an electrically conductive gel that contacts the patient's skin and conducts to the electrode electrical signals that are present at the skin. The patient 140's heart produces an electrical signal that is referred to as an ECG waveform, and which may also be referred to herein as an ECG signal, an ECG lead signal, an ECG, or ECG data.

Specifically, electrodes 150 include four limb electrodes, including electrode 120 placed on a right arm of patient 140 (and therefore conventionally referred to as RA), electrode 122 placed on a right leg of patient 140 (and therefore conventionally referred to as RL), electrode 138 placed on a left arm of patient 140 (and therefore conventionally referred to as LA), and electrode 124 placed on a left leg of patient 140 (and therefore conventionally referred to as LL). The four limb electrodes may be used to measure the electrical potential of the heart along six distinct axes intersecting the heart, where each of the electrical potentials measured along a distinct axis is referred to as a lead. The six leads which the four limb electrodes are configured to measure are referred to as I (which generates the electrical potential difference between electrode 138 and electrode 120), II (which generates the electrical potential difference between electrode 124 and electrode 120), III (which generates the electrical potential difference between electrode 124 and electrode 138), aVR (which generates the electrical potential difference between electrode 120 and the average of electrodes 124 and 138), aVL (which generates the electrical potential difference between electrode 138 and the average of electrodes 120 and 124), and aVF (which generates the electrical potential difference between electrode 124 and the average of electrodes 120 and 138).

Further, electrodes 150 has six chest electrodes, including electrode 126 (conventionally referred to as V1), electrode 128 (conventionally referred to as V2), electrode 130 (conventionally referred to as V3), electrode 132 (conventionally referred to as V4), electrode 134 (conventionally referred to as V5), and electrode 136 (conventionally referred to as V6). The six chest electrodes, in conjunction with the limb electrodes, are configured to measure electrical potentials through six distinct axes intersecting the heart in a horizontal plane (that is, a plane perpendicular to the plane in which the six limb leads are measured). Specifically, the six leads which the six chest electrodes are configured to measure are referred to as V1 (which generates the potential difference between electrode 126 and the average of electrodes 120, 124, and 138), V2 (which generates the potential difference between electrode 128 and the average of electrodes 120, 124, and 138), V3 (which generates the potential difference between electrode 130 and the average of electrodes 120, 124, and 138), V4 (which generates the potential difference between electrode 132 and the average of electrodes 120, 124, and 138), V5 (which generates the potential difference between electrode 134 and the average of electrodes 120, 124, and 138), and V6 (which generates the potential difference between electrode 136 and the average of electrodes 120, 124, and 138).

ECG data acquired by data acquisition module 118 may be transmitted to ECG processing system 102 for storage, and processing (signal filtering, normalization, noise suppression, etc.). ECG data processing system 102 may further be configured to automatically diagnose ECG data acquired by data acquisition module 118 by executing one or more operations of one or more of the methods herein disclosed. ECG processing system 102 includes a processor 104, and non-transitory memory 106, where processor 104 may read instructions from non-transitory memory 106 to execute one or more operations of one or more of the methods stored therein. ECG processing system 102 is further communicatively coupled to display device 114, and user input device 116, which may enable a user to see, and interact with, data within ECG processing system 102, respectively.

ECG processing system 102 includes the processor 104 configured to execute machine readable instructions stored in non-transitory memory 106. Processor 104 may be single core or multi-core, and the programs executed thereon may be configured for parallel or distributed processing. In some embodiments, the processor 104 may optionally include individual components that are distributed throughout two or more devices, which may be remotely located and/or configured for coordinated processing. In some embodiments, one or more aspects of the processor 104 may be virtualized and executed by remotely-accessible networked computing devices configured in a cloud computing configuration.

Non-transitory memory 106 may store a cycle length calculation algorithm 110 and an ECG data module 112. Cycle length calculation algorithm 110, which is further described as method 200 in FIG. 2, may in turn comprise a method 300 as shown in FIG. 3 for learning segments of ECG data waveform morphology to use as templates, and a method 500 as shown in FIG. 5 for using learned templates to identify and mark ECG signals, as well as calculate cycle length. Results determined by method 200 are output as visual indicators to an operator, such as, for example, a tick mark placed at a heartbeat and the cycle length value displayed adjacent to the corresponding tick mark, allowing the result to be observed by the operator.

Non-transitory memory 106 further includes ECG data module 112, which includes ECG data acquired by one or more data acquisition modules or ECG systems. In some embodiments, ECG data module 112 includes data acquired by ECG system 100. In some embodiments, ECG data module 112 may store ECG data acquired through communicative coupling with one or more data sources other than ECG processing system 102. ECG data stored within ECG data module may be organized according to one or more organizational schemes, or configured into one or more data structures known in the art of data storage. In some embodiments, ECG data may be stored in ECG data module by indexing the ECG data according to lead set, thereby enabling rapid look-up of ECG data by lead set. For example, a query for ECG data comprising the six chest leads may be executed by ECG processing system 102 by evaluating an index for each ECG data set included therein, and returning each ECG data set with an index indicating that the ECG data set includes the six chest leads.

In some embodiments, the non-transitory memory 106 may include components disposed at two or more devices, which may be remotely located and/or configured for coordinated processing. In some embodiments, one or more aspects of the non-transitory memory 106 may include remotely-accessible networked storage devices configured in a cloud computing configuration.

ECG system 100 further includes user input device 116. User input device 116 may comprise one or more of a touchscreen, a keyboard, a mouse, a trackpad, a motion sensing camera, or other device configured to enable a user to interact with and manipulate data within ECG processing system 102. As an example, user input device 116 may enable a user to make a selection of ECG data to diagnose.

Display device 114 may include one or more display devices utilizing virtually any type of technology. In some embodiments, display device 114 may comprise a computer monitor, and may display unprocessed and processed ECGs data. Display device 114 may be combined with processor 104, non-transitory memory 106, and/or user input device 116 in a shared enclosure, or may be peripheral display devices and may comprise a monitor, touchscreen, projector, or other display device known in the art, which may enable a user to view ECG data and/or interact with various data stored in non-transitory memory 106.

It should be understood that ECG system 100 shown in FIG. 1 is for illustration, not for limitation. Another appropriate ECG processing system, for example, an ICEG system, may include more, fewer, or different components.

Analyzing ECG data to identify heartbeats and determine cycle length is conventionally based on visually identifying recurrent signals of ECG data based on a signal amplitude, or deflection, and manually calculating cycle lengths as a distance between peak signal amplitudes. This method may be reliable for ECG data where the shape, or waveform morphology, of a recurrent signal has distinct peak amplitudes with a gap in time therebetween. Distinct signals with visually discernable peak amplitudes is common in healthy hearts (e.g., a heart not experiencing arrhythmia). Still, signals not caused by a heartbeat may be present in the ECG data due to noise from the ECG system, patient muscle movement, or catheter/electrode movement, which may generate an erroneous signal.

For patients with heart disease, such as arrhythmia, signals may be smaller and/or not as distinct compared to ECG data from a healthy heart. FIG. 8 shows example ECG displays 800 including graphs of ECG data for a healthy heart (graph 802) and for a heart experiencing arrhythmia (graph 804), as may be collected by the ECG system of FIG. 1. The ECG system may generate 2D data of electrical potential relative to time, wherein electrical potential is plotted along a y-axis, and time is plotted along an x-axis in milliseconds. A set of reference axes 801 are provided, indicating the x-axis and the y-axis.

Graph 802, which shows ECG data for a healthy heart, includes plot 806 with a first signal 808, a second signal 810, a third signal 812, and a fourth signal 814, where a signal is a peak in the electrical potential indicating a heartbeat. As each signal has the same waveform morphology, which includes characteristics of an ECG signal such as shape and amplitude of the electrical potential, the four signals 808-814 may be recurrent signals. One or more of the characteristics of waveform morphology, alone or in combination, may aid in diagnosing and distinguishing heart disease, such as arrhythmia, by identifying signals as recurrent and therefore potential or candidate heartbeats, as opposed to a signal that originates from a source other than electrical potential of a heart, such as noise from patient muscle movement, electrode movement, etc. Additionally, a signal peak for the first signal 808 occurs at time t1, a signal peak for the second signal 810 occurs at t2, and a signal peak for the third signal 812 occurs at t3. The durations, or cycle lengths, between t1 and t2 and between t2 and t3 are equal, indicating the signals are evenly spaced and the heart has a normal, healthy rhythm.

Graph 804, which shows ECG data for a heart with arrhythmia, includes plot 816 with a first signal 818, a second signal 820, a third signal 822, and a fourth signal 824. As each signal has the same waveform morphology, the four signals 816-824 may be recurrent signals. Additionally, a signal peak for the first signal 818 occurs at time t1, a signal peaks for the second signal 820 occurs at t2, and a signal peak for the third signal 822 occurs at t3. The duration, or cycle length, between t1 and t2 is less than the cycle length between t2 and t3, indicating the signals are not evenly spaced and the heart has an irregular rhythm, herein referred to as arrhythmia.

If signals are visually identified by an operator to calculate cycle length, differentiating a true signal from a signal caused by noise may be time consuming and workflow disruptive. A method for more accurately diagnosing heart disease relative to the conventional method of identifying recurrent signals of ECG data based on signal waveform morphology is desired. In one example, automatic identification of recurrent signals may be desirable to increase both an accuracy and an efficiency of identification.

Automated determination of atrial fibrillation cycle length values is described by MacAdam et al. in U.S. Pat. No. 8,903,478, where cycle length is identified, ECG data is annotated with cycle length values, and maps are created which coordinate location information of intracardiac catheter electrodes with cycle length values to guide ablation therapy. The method described by MacAdam et al. includes identifying signals for which a cycle length is calculated by using a filter to digitally process the ECG signals and identify each new cycle in a configured time interval. The filter may be trained against a set of known data so that the filter responds to a prescribed electrical signal pattern, such as a predetermined signal amplitude, corresponding to a known recurrent signal. An operator may also manually identify the beginning or end of an ECG segment of interest over which cycle length determinations are made. While this method partially automates identification of ECG signals and determination of cycle length, further automation is possible.

By relying on identification of an ECG signal based on a predetermined amplitude in order to calculate a cycle length, detection of irregular signals as true signals may be overlooked. As a result, a patient with heart disease may be misdiagnosed. Thus, accuracy of identification may be constrained by a capability of the identification method. Furthermore, the irregular signals may be due to other factors aside from heart disease, including noise, operator error, etc., which may confound efficient differentiation between a heartbeat and the other, non-cardiac related factors.

The inventors herein have developed a method for identifying ECG signals by continuously learning waveform morphology of ECG data and calculating a cycle length for an ECG signal in real-time using machine learning. Continuous learning in real-time includes applying the method to all incoming ECG data while the ECG data is being collected. The method identifies potential recurrent signals based on recurrent waveform morphology of the present ECG data and independent of a predetermined amplitude or known signal morphology preemptively programmed into the system. Correlation calculations are performed among potential recurrent signals to identify waveform morphology with true recurrence, and templates are generated based on the recurrent waveform morphology to compare against ECG data in real-time to continuously identify recurrent signals. Additionally, the templates are continuously updated to reflect changes in ECG data waveform morphology, which may be due to shifting electrodes/catheters or treatment of the heart disease. Heartbeats may be identified, enabling calculation of heartbeat cycle length which may be displayed at a user interface. As a result, heartbeats in patients with heart disease may be rapidly and accurately identified in real-time using machine learning, and indicated to an operator, as well as cycle lengths of the identified heartbeats.

Turning to FIG. 2, a high level method 200 for calculating a cycle length of a recurrent signal of ECG data is shown. In some embodiments, method 200, and the other methods described herein, may be executed by the ECG system 100 illustrated in FIG. 1.

Method 200 begins at 202, where ECG data is acquired by an ECG or ICEG system. ECG data may include one or more lead signals/waveforms, indicating an electrical activity of a heart over time. In some embodiments, acquiring ECG data may include measuring cardiac activity and generating an electrocardiogram (ECG). In some embodiments, acquiring ECG data may include measuring an electrical potential at one or more intracardiac leads using one or more catheters configured with multiple electrodes and disposed at various locations within the heart. In some embodiments, acquiring ECG data may include measuring a combination of twelve lead ECG and ICEG. The ECG data acquired at 202 may generate 2D data of electrical potential through time, wherein electrical potential is plotted along the y-axis, and time is plotted along the x-axis. In some embodiments, the acquired ECG data may be stored in one or more formats, including SCP-ECG, DICOM-ECG, HL7 aECG, and other storage formats known in the art of ECGs. Acquisition of ECG data at 202 may occur under various conditions, such as when the patient is resting or in an ambulance.

At 204, the ECG data is filtered to remove excess noise. Noise may include signal activation from sources other than the patient heartbeat, which may include patient movement, catheter or electrode movement, machine noise, etc., all of which may be inaccurately registered as a signal if not filtered out from the ECG data. An ECG processor, such as the processor 104 of FIG. 1, may filter the ECG data. The processor may include, in one example, a band pass filter. In one example, the band pass filter may operate between 15 Hz and 60 Hz.

Once ECG data has been filtered, at 206, the method 200 learns segments of waveform morphology of the ECG data to be used as templates. This method is further described below with reference to method 300 of FIG. 3.

After templates have been learned based on segments of waveform morphology from filtered ECG data at 206, the method continues to 208. At 208, the templates are used to identify and mark recurrent signals of ECG data in real time, where recurrent signals are signals with the same waveform morphology as the templates and each other, and may therefore be more likely to be a true heartbeat as opposed to a signal caused by noise or other non-heartbeat activation. Additionally, a cycle length for each identified recurrent signal is calculated. This method is further described below with reference to method 500 of FIG. 5.

The method 200 may occur continuously in parallel and independently at each of the ECG channels (e.g., each of the twelve lead ECG and/or ICEG channels). The method 200 is repeated indefinitely while a cycle length calculation algorithm, such as the cycle length algorithm 110 of FIG. 1, is operational, thereby allowing the system to continuously update the templates and calculate cycle lengths for dynamically changing signals.

Turning to FIG. 3, a method 300 is shown for learning segments of the ECG waveform morphology to be used as templates. Method 300 may be executed as part of method 200, as indicated at 206. The templates learned in method 300 may be applied to method 500 of FIG. 5 to identify and mark recurrent signals of ECG data and determine cycle lengths of the recurrent signals, as described below. In some embodiments, method 300 may be executed by the cycle length calculation algorithm. Method 300 occurs continuously and independently on each channel of the ECG system 100 and repeats indefinitely while the ECG system is collecting data, as described at 202 of method 200. The continuous implementation of method 300 allows learning of the ECG waveform morphology to continue as new data is collected, allowing the method to continuously update the templates for dynamically changing signals.

Method 300 of FIG. 3 will be described in parallel with FIGS. 4A-C, which illustrate example results of method 300 when applied to ECG data. FIG. 4A shows examples of incoming and filtered ECG data, FIG. 4B shows a learning template derived from the filtered ECG data of FIG. 4A, and FIG. 4C shows a final template derived from the learning template of FIG. 4B. The ECG data, including the learning template and the final template, may be displayed as 2D data of electrical potential relative to time, wherein electrical potential is plotted along a y-axis, and time is plotted along an x-axis in milliseconds. A set of reference axes 401 are provided for FIGS. 4A-C, indicating the x-axis and the y-axis. The time markers of each of the graphs of FIGS. 4A-C may or may not occur concurrently. For example, 100 ms on graph 404 of FIG. 4B may occur at, before, or after 100 ms on graph 402 of FIG. 4A.

Returning to FIG. 3, at 302, method 300 captures a segment of filtered ECG data for a time t1. In one example, t1 is two seconds. In another example, t1 may be greater than or less than two seconds. Prior to method 300, incoming ECG data is amplified and digitized, for example, by the data acquisition module 118 of FIG. 1, and filtered, for example, by the ECG processor 104 of FIG. 1, to generate filtered ECG data. Graph 402 of FIG. 4A shows an example comparison of incoming and filtered ECG data. The incoming ECG data (plot 412) is the data collected by the ECG system, for example the twelve lead ECG, the ICEG, or a combination thereof, as described with reference to FIG. 1. The filtered ECG data (plot 410) is obtained by filtering incoming ECG data, for example, as described at 204 of method 200 of FIG. 2. The graph 402 shows a first signal 403 occurs between approximately 0 and 150 ms and a second signal 405 occurs between 150 and 300 ms.

Furthermore, at 302, method 300 includes operations for processing the segment as a learning template. A learning template is a signal of ECG data that may be a heartbeat and will be further processed by method 300 to determine correlation with other learning templates to generate a final template used to identify heartbeats, as described below. For example, to process the segment as a learning template, at 304, a maximum value and a minimum value of electrical potential of a signal of the segment of filtered ECG data are determined. In FIG. 4A, the maximum and the minimum values for the first signal 403 of filtered ECG data 410 are represented by horizontal lines 420 and 422, respectively. At 306 of FIG. 3, a first threshold range is defined using the maximum and minimum values determined at 304, where the first threshold range is a span of values between the minimum and the maximum values. When a signal crosses the maximum or the minimum values, the first threshold range may be exceeded. For example, the first threshold range may be defined by a maximum value of +1 mV and a minimum value of −1 mV. In the example of FIG. 4A, values between the maximum value line 420 and minimum value line 422 represent the first threshold range.

Method 300 includes generating a threshold number of learning templates, to be further described below at 312. In the exemplary embodiment, where t1 is equal to two seconds and the threshold number of learning templates is five, it may take a minimum duration of ten seconds for method 300 to repeat, as further described below, to learn a first final template. To identify potential instances of recurrent signals during the ten seconds in which the first final template is being learned, the method includes, after the first threshold range is defined at 306, confirming if there are greater than zero final templates stored in the system at 326. If there are greater than zero final templates, then the system has already identified at least the first final template to which ECG signals can be compared in method 500 of FIG. 5. Method 300 continues to 308 to identify periods of interest in the segment where the ECG signal crosses an upper or lower bound of a second threshold range, to be further described below.

Returning to 326, if there are not greater than zero final templates, that is, there are no final templates stored in the system, the method 300 continues to 328 to determine if the signal within the segment captured at 302 crosses an upper or lower bound of a third threshold range, where the third threshold range is a first percentage that is greater than a second percentage, described below at 308. In one example, the first percentage is 70%. For example, if an upper bound of the first threshold range is 1 mV set by the maximum value and a lower bound of the first threshold range is −1 mV set by the minimum value at 306, wherever the signal crosses +0.7 mV or −0.7 mV, the third threshold range is exceeded.

In another example, the first percentage may be greater than or less than 70%, but still greater than the second percentage. In the example of FIG. 4A where the first percentage is 70%, horizontal line 424 represents the upper bound of the third threshold range and horizontal line 425 represents the lower bound of the third threshold range. If the signal the upper or lower bound of the third threshold range at 328 of FIG. 3, the signal is defined as a beat at 330. At 332, a tick mark is placed, then method 300 returns to start. In one example, the tick mark is placed at the highest point of the signal amplitude. In another example, the tick mark is placed at the point where the signal crosses the upper or lower bounds of the third threshold range. In a third example, the tick mark is placed at the middle point of the signal, where the middle point may be one second when t1 for capturing the ECG segment is two seconds. If the signal does not cross the upper or lower bounds of the third threshold range, then method 300 returns to the start without placing a tick mark.

The processes described at 326-332 of method 300 for identifying a signal before the first final template is identified may be a strategy to initiate detection of beats early in ECG data collection, e.g., after two seconds instead of waiting ten seconds until the first final template is identified, when t1 is two seconds. The third threshold range encompasses a wider range of values than the second threshold range so as to deter erroneous identification of noise as a beat. However, beats identified using the process described at 326-332 of method 300 may be less accurate than method 500, described below. The position of the tick mark placed during method 500 depends on methods 300 and 500, which include performing correlation percent calculations, generating a final template based on correlation percent calculations and exceeding multiple signal and correlation percent thresholds, to be further described below, while the position of the tick mark placed at 332 depends on exceeding a single signal threshold, e.g., crossing the upper or lower bounds of the third threshold range. Therefore, a more robust method for determining tick mark position is used in method 500 for the tick mark placed compared to the tick mark placed at 332 of method 300, which may result in greater placement accuracy for the tick marks displayed in method 500.

Returning to 308, a period or periods of interest are identified in the segment where the signal crosses the maximum or minimum values of the second threshold range. The period of interest may be approximately 320 ms, in one example. In another example, the period of interest may be greater than or less than 320 ms, depending on an operator-defined time for which to capture the period of interest. In another example, the period of interest may be a function of signal type, where, depending on if the ECG data is from a surface ECG or ICEG, a duration of the period of interest may be adjusted on a per channel basis, as signals from an ICEG channel may be more localized and therefore of shorter duration compared to signals from a ECG channel. The second threshold range may be defined by the second percentage of the first threshold range. In the exemplary embodiment, the second percentage may be 40% of the first threshold range. For example, if an upper bound of the first threshold range is 1 mV set by the maximum value and a lower bound of the first threshold range is −1 mV set by the minimum value, wherever the signal crosses a value that is 40% of the upper or lower bounds of the first threshold range, for example, +0.4 mV or −0.4 mV, the period of interest is identified. In another example, the second percentage may be greater than or less than 40%. In the example of FIG. 4A where the first percentage is 40%, horizontal line 426 represents an upper bound of the second threshold range and horizontal line 427 represents a lower bound of the second threshold range. At 310, the period of interest is saved in the system memory, for example, the non-transitory memory 106 of FIG. 1, as a learning template.

Graph 404 of FIG. 4B shows a learning template 414, which may be derived from the filtered ECG data 410 of FIG. 4A. The learning template 414 has a total length of 240 ms and includes a signal between approximately 100 and 150 ms, as well as baseline electrical potential before and after the signal.

At 312 of FIG. 3, method 300 includes determining if a target number of learning templates have been saved. In one example, the target number of learning templates is five. In another example, the target number of learning templates may be greater than or less than five. If the target number of learning templates have not been saved, the method 300 returns to 302 and repeats 302-310 until the target number of learning templates are saved in the system. Returning to 312, if the target number of learning templates has been saved, the method 300 continues to 314 to perform a correlation percent calculation of the learning templates against each other. Various methods known to those skilled in the art may be used by the method 300 to determine a correlation percentage among each of the target number of learning templates.

If, at 316, a single learning template does not have a correlation percentage above a fourth threshold with regards to at least a first number of other learning templates of the target number of learning templates, method 300 returns to the start and continues to learn learning templates. In the present example, the first number of learning templates with which the single learning template may correspond is three of the target number of five learning templates. In one example, the fourth threshold may be a correlation percentage of 90%. In another example, the fourth threshold may be greater than or less than 90%. If the learning template does correlate with at least three other learning templates above the fourth threshold at 316, method 300 continues to 318 to define the learning template as a final template.

The final template, which may have a length of 320 ms in one example as described above, may be trimmed to a section of interest at 320 of method 300. In one example, the section of interest is a middle portion of the final template, which may be the middle 160 ms of the total final template length. The section of interest may encompass a point where the signal of the final template is greater than the upper bound of the second threshold range, as determined at 308. Trimming the final template may reduce noise and isolate a beat in the signal.

In the example of FIG. 4B, the learning template 414 is confirmed to have an above threshold correlation with at least three other learning templates, as determined at 316 of method 300, and the learning template 414 is defined as a final template at 318. Graph 406 of FIG. 4C shows the learning template 414 of FIG. 4B trimmed to the final template 416. The learning template 414 is trimmed, as described above with reference to 320 of method 300, to the section of interest, for example, the region between approximately 50 to 170 ms of FIG. 4B. The final template length for plot 416 of FIG. 4C is 120 ms and the final template is stored for use in method 500 of FIG. 5 to identify and mark occurrences of recurrent signals. In this way, when the final template is used in method 500 of FIG. 5 to identify instances of recurrent signals, method 500 may more accurately identify points of correlation between the final template and points of the ECG signal representing a heartbeat, compared to using an untrimmed final template.

Method 300 includes confirming if a target number of final templates have been identified and stored in the system at 322. In one example, the target number of final templates may be three. In another example, the target number of final templates may be greater than or less than three. The system may store the target number of final templates synchronously. If there are less than the target number of final templates, the method 300 returns to the start and segments of the ECG waveform morphology continue to be learned to be used as final templates. If the target number of final templates are stored, method 300 at 324 replaces a stored final template having a least number of votes compared to the other stored final templates with a newly generated final template. The vote counting system will be described below with respect to method 500 of FIG. 5. Method 300 returns to the start and method 300 repeats indefinitely while the system collects ECG data. Additionally, method 300 returns to method 200 of FIG. 2, where the final templates generated by method 300 may be used by method 500 of FIG. 5 to identify and mark ECG signals and calculate cycle length.

In this way, method 300 identifies and learns segments of the incoming ECG waveform morphology to be used as a template against which to compare incoming ECG data to identify recurrent signals. These recurrent signals may indicate an irregular heartbeat and help physicians identify heart disease or other problems with heart function. By identifying at least a target number of learning templates from which to identify a final template, and then trimming the final template to a section of interest, method 300 may produce a final template that represents an average waveform morphology that has recurrence to which incoming ECG data may be compared. Prior to identifying a first final template, method 300 may identify and mark a potential beat without the use of a final template, so as to allow cycle length calculation, to be further described below, as early as from the first two seconds during which method 300 is operational, instead of waiting until the final template is generated after a minimum ten seconds, in one example. As method 300 repeats indefinitely while the system collects ECG data, final templates are continuously generated using method 300 and the three final templates stored by the system are replaced to reflect incoming waveform morphology. The final templates are used in the method 500 of FIG. 5 to identify and mark ECG signals, ultimately allowing calculation of cycle length for ECG signals.

Turning to FIG. 5, a method 500 is shown for using the learned final templates to identify and mark recurrent signals, and calculate cycle lengths of recurrent signals. Method 500 may be executed as part of method 200, as indicated at 208 of method 200. In some embodiments, method 500 may be executed by the cycle length calculation algorithm 110, implemented by ECG system 100 of FIG. 1. Method 500 occurs continuously and independently on each channel of the ECG system 100 and repeats indefinitely while the ECG system 100 is collecting data, for example, at 202 of method 200. Method 500 allows identification and marking of recurrent signals, as well as cycle length calculation, to continue as new data is collected, allowing the method to calculate cycle length for dynamically changing signals.

Method 500 of FIG. 5 will be described in parallel with FIGS. 6A-B, which show examples of incoming and filtered ECG data annotated with markers showing points of interest which may correspond to steps of method 500, and a final template, respectively. The ECG data, including the final template, may be displayed as 2D data of electrical potential relative to time, where electrical potential is plotted along a y-axis, and time is plotted along an x-axis in milliseconds. A set of reference axes 605 are provided for FIGS. 6A-B, indicating the x-axis and the y-axis. The time markers of each of the graphs of FIGS. 6A-B may not represent concurrent times. For example, 100 ms on graph 603 of FIG. 6B may occur at, before, or after 100 ms on graph 601 of FIG. 6A.

Returning to FIG. 5, at 502, method 500 includes scanning filtered ECG data for instances where a signal crosses the upper or lower bound of the second threshold range, as defined above with respect to 306 of method 300 of FIG. 3. Prior to method 500, incoming ECG data has been amplified and digitized, for example, by the data acquisition module 118 of FIG. 1, and filtered by the ECG processor 104 of FIG. 1. Graph 601 of FIG. 6A shows an example comparison of incoming and filtered ECG data. The incoming ECG data (plot 606) is the data collected by the ECG system, for example the twelve lead ECG, the ICEG, or a combination thereof, as described with reference to FIG. 1. The filtered ECG data (plot 610) is the result of filtering the incoming ECG data, for example, as described at 204 of the method 200 of FIG. 2. The graph 601 shows a first signal 630 occurring between approximately 50 and 400 ms, and a second signal 632 between 750 and 1000 ms, the second signal 632 continuing beyond what is shown on the graph 601. The first and the second signals 630, 632 may be recurrent signals, as will be further described by the description of method 500.

In the example of FIG. 6A, the second threshold range is 40% of the first threshold range, where the first threshold range is defined based on maximum and minimum values of electrical potential of the incoming data segment. The maximum and minimum values for the first signal 630 are shown by horizontal lines 634 and 636, respectively, and the upper and lower bounds of the second threshold range are shown by horizontal line 638 and 639, respectively. Where the amplitude of the first signal 630 is greater than horizontal line 638, the first signal 630 crosses the upper bound of the second threshold range.

At 504, if the signal does not cross the upper or lower bounds of the second threshold range, the method 500 returns to 502 and continues to scan the incoming ECG data. If the signal does cross the upper or lower bounds of the second threshold range at 504, the method 500 proceeds to 506 to capture a candidate beat, e.g., a possible heartbeat, which encompasses the signal at the point where the signal exceeds the first threshold range, as well as a region equal to twice the length of the final template. For example, if the final template is 120 ms, the candidate beat is captured for a duration of 240 ms, with the point at which the signal exceeds the signal threshold centered in the candidate beat interval.

In the examples of FIGS. 6A-B, graph 603 shows a final template 624, which may be generated by method 300. The first signal 630 of graph 601 crosses the upper bound of the second threshold range 638 at marker 612, and the first signal 630 is identified as part of a candidate beat. The length of final template 624 is 150 ms, therefore, ECG data is captured for 150 ms on either side of marker 612, so the captured candidate beat 608 is 300 ms and ranges from 100 ms to 400 ms, with the marker 612 centered in the total length.

Returning to FIG. 5, at 508, a correlation percent calculation of the candidate beat is performed with each of the three final templates defined in method 300 using the correlation method described above with respect to 314 of method 300 of FIG. 3.

At 510, method 500 includes determining if the correlation of the candidate beat with each of the three final templates exceeds a fifth threshold. In one example, the fifth threshold may be a correlation percentage of 70%. If the correlation percentage does not exceed the fifth threshold at 510, method 500 returns to the start and continues to scan incoming ECG data for signals that correlate with the final templates. If correlation between the candidate beat and at least one of the three final templates is greater than 70%, the candidate beat may be confirmed as an actual beat and method 500 proceeds to 512 to process the beat. For example, starting at 100 ms on the graph 601, method 500 attempts to match ECG waveform morphology of the three final templates to the waveform morphology of the candidate beat 608 by performing a correlation percent calculation. In the example of FIGS. 6A-B, the candidate beat 608 is confirmed to exceed the fifth threshold and therefore correlate with the final template 624. A correlation percent marker 614, that is, the percentage that the final template correlates with the candidate beat 630, is displayed at the end point of the captured candidate beat length 608 at 400 ms. The amplitude of the correlation percent marker 614 indicates the correlation percent value. In the example of FIG. 6A, the correlation percent is approximately 100%.

Returning to method 500, processing the beat at 512 includes placing a tick mark, calculating a cycle length, and giving a vote to the correlating final template. At 514, a tick mark is placed on the ECG data at the point of greatest correlation between the beat and the final template that achieved the highest correlation percentage, compared to the other two final templates. In another example, the tick mark may be placed at the highest point of the signal amplitude. In one example, the tick mark may be placed at the middle time point of the template.

At 516, the cycle length is calculated for the beat. The cycle length may be calculated as a distance from the tick mark of the present beat to the tick mark of an immediately previous beat. If method 500 is implemented to calculate a first cycle length, the tick mark of the previous beat may be that of the beat identified without use of a final template, as described with respect to 326-332 of method 300 of FIG. 3. If method 500 is used to calculate a cycle length that is not the first cycle length, the tick mark of the previous beat may be a tick mark determined using method 500.

For the example shown in FIGS. 6A-B, the final template 624 is found to match the candidate beat 608 starting at marker 602, and the candidate beat 608 is considered a beat 608. As described with respect to 514 of method 500, a tick mark 604 is placed at the point of the beat 608 where the final template, in this case 624, has the highest correlation percentage with the beat 608. Though not shown in FIG. 6A, the final template can be considered to be overlaid on the filtered ECG data 610, allowing the tick mark to be placed on the filtered ECG data and therefore the incoming ECG data 606 at the point of most correlation between the final template and the beat 608. A cycle length is calculated for the beat 608 using the tick mark 604 and a tick mark of a previous beat, not shown.

Additionally, at 518, the final template which was found to have the highest correlation percentage with the beat, compared to the other two templates, is given a vote to show that the final template in question had a sufficient correlation with a recurrent signal to be deemed a match and was therefore an accurate example of a beat. When final templates are replaced in 324 of method 300 of FIG. 3, the final template with the least number of votes is replaced.

After the operations of 512 are completed, including placing a tick mark at 514, calculating cycle length at 516, and giving the correlating final template a vote at 518, method 500 includes waiting for a duration t2 of a refractory period at 520 before method 500 returns to the start and repeats. In the exemplary embodiment, the refractory period is 160 ms. It is known in the art that a maximum heart rate is approximately 310 bpm, which includes a refractory period of approximately 160 ms. Waiting until the refractory period is over to repeat method 500 may reduce noise and allow the method 500 to identify a signal that is more likely to be a recurrent signal instead of noise during the refractory period. In the example shown in FIGS. 6A-B, the duration t2 is shown by duration 635. The refractory period begins at the marker 612 where the signal 630 exceeded the upper bound of the second threshold range 638. In the example of FIG. 6A, the refractory period is 200 ms, and ends at the marker 616.

As method 500 is continuously applied to ECG data, after duration t2 has passed, method 500 returns to the start and repeats indefinitely while the system collects ECG data. Furthermore, method 500 continually scans filtered ECG data, e.g., ECG data may not be segmented and instead scanned as a continuous stream of incoming data to identify where the signal may cross the upper or lower bounds of the second threshold range, as opposed to scanning segments of data captured for time t1, as described above at 302 of method 300.

In the example of FIGS. 6A-B, method 500 repeats for the second signal 632, where an upper bound of a second threshold range 640 is exceeded at 622, the final template 624 is found to match a candidate beat 642 including the second signal 632 starting at marker 618, and the tick mark is placed at 620. As described with respect to 516 of method 500, a cycle length is calculated for the beat 632 as a distance between tick marks 604 and 620. In the example of FIG. 6A, a cycle length 644 for beat 632 is approximately 700 ms.

Additionally, method 500 returns to method 200 of FIG. 2, where method 200 may return to the start and repeats to continue determining cycle lengths as long as the cycle length calculation algorithm of the ECG system is operational.

The filtered ECG data 610 and time markers 602, 612, 614, 616, 618, and 622 of graph 601, and the final template 624 of graph 603 are shown in FIGS. 6A-B as examples to show the operations of method 500, and are not displayed at a display device, such as the display device 114 of FIG. 1. Each time marker 602, 604, 612, 616, 618, 620, and 622 indicates an operation of method 500, and has an arbitrary amplitude to allow a viewer to differentiate between time markers. For example, the marker 602 and the marker 618 have a same, first amplitude and both represent a start of the correlating final template. Further, the marker 612 and the marker 622 have a same, second amplitude that is different from the first amplitude of markers 602, 618. The incoming ECG data 606 is shown on the display device, as well as tick marks 604, 620 placed at the center of the final template that correlates with the current signal. Although the tick marks 604, 620 of FIG. 6A appear to be placed in the center of the signal peak, this placement may vary with respect to the x-axis, as the tick mark is placed according to the center of the final template that matches the filtered ECG data which may not align exactly with the incoming ECG data. In other examples, the tick marks may be placed at the highest point of the signal amplitude or at the point where the signal has greatest correlation with a final template.

In this way, method 500 uses the final templates defined by method 300 to identify, mark, and calculate cycle lengths of recurrent signals. These recurrent signals may indicate an irregular heartbeat and help physicians identify heart disease or other problems with heart function. By performing correlation calculations among a candidate beat and each of three final templates, and placing a tick mark where the candidate beat correlates above a fifth threshold with one of the three final templates, method 500 may identify a recurrent signal that is a heartbeat and not noise generated by a source other than the patient's heart. Calculating a cycle length for each recurrent signal identified as a beat, marking the beat, and displaying the cycle length on a display device may allow physicians to identify irregular heartbeats which may aid in the diagnosis of heart disease, such as arrhythmia.

FIG. 7 shows an example ECG display with five channels displayed corresponding to electrodes V1, V2, V3, V4, and V5, as described with reference to FIG. 1. A letter M at the beginning (e.g., far left) of each channel display indicates the cycle length calculation algorithm is active. In the example of FIG. 7, tick marks are placed at a time relative to the final template, for example, the middle of the duration of the final template. Each channel displays incoming ECG data with a tick mark at each recurrent signal, and the cycle length, in milliseconds, of the preceding beat displayed to the left of the tick mark. For example, the cycle length 615 displayed at channel V1 is the cycle length for a beat 702 and the cycle length 786 is the cycle length for a beat 704.

In this way, final templates generated based on learned ECG data waveform morphology are used to identify and mark recurrent signals as heartbeats, calculate a cycle length of each heartbeat, allowing the marked heartbeat with the corresponding cycle length to be displayed at a user interface.

A set of learning templates are generated based on segments of ECG data where a signal amplitude exceeds a first signal threshold, and a correlation percentage of waveform morphology among the set of learning templates is calculated to generate a final template based on a first correlation threshold. A target number of final templates are generated and the final templates are used to identify a recurrent signal in real-time based on a second correlation threshold.

Continuously learning segments of the ECG waveform morphology to generate templates and using the templates to identify and mark recurrent signals may occur in parallel and independently on each channel of the ECG system. Additionally, the cycle length of each recurrent signal may be displayed at the respective recurrent signal in real time on a user interface. Performing these methods continuously allows the set of final templates to be updated and continue to identify recurrent signals as the ECG waveform morphology changes. The ECG waveform morphology may change for a number of reasons, including intentional or unintentional movement of the cathodes/electrodes, or if changes are made to heart tissue or electrical activity due to cardiac treatment, for example, operation, in real time.

The technical effect of using machine learning to continuously identify recurrent signals in ECG data based on waveform morphology is that heartbeats and corresponding cycle lengths may be identified and estimated, respectively and concurrently, in real-time, allowing an operator to efficiently differentiate between heartbeats and non-cardiac signals as the ECG data is generated. Additionally, as the method occurs in real-time and continuously outputs results, processing efficiency may be improved.

The disclosure also provides support for a method for an electrophysiology study, comprising continuously learning waveform morphology of electrocardiogram data in real-time to identify recurrent signals, indicating a heartbeat for each of the recurrent signals at a user interface as the recurrent signals are identified, estimating a cycle length based on the heartbeat, and displaying the cycle length for each heartbeat at the user interface.

The disclosure also provides support for a method for an electrophysiology study, comprising continuously learning waveform morphology of electrocardiogram data in real-time to establish a plurality of standardized electrocardiogram (ECG) patterns based on the ECG data, selecting signals of real-time ECG data based on correlation among real-time ECG data and at least one pattern of the plurality of standardized ECG patterns, determining a cycle length of each signal of the signals of real-time ECG data, and displaying the cycle length for the respective signal at a user interface.

The disclosure also provides support for a method for an electrophysiology study, comprising sensing patient electrocardiogram (ECG) data, continuously selecting standardized ECG waveform patterns based on correlations within the sensed patient ECG data, when the correlation is greater than a non-zero threshold, processing the sensed patient ECG data to identify a point of maximum correlation among the sensed patient ECG data and standardized ECG waveform patterns, determining a cycle length based on the identified point in real-time, and displaying the cycle length for the respective identified point at a user interface.

The disclosure also provides support for a method for an electrophysiology study, comprising establishing a plurality of standardized electrocardiogram (ECG) patterns based on real-time sensed ECG data, selecting signals of the real-time sensed ECG data based on a correlation between the real-time sensed ECG data and at least one pattern of the plurality of standardized ECG patterns, determining a cycle length of each of the signals of the real-time ECG data, and displaying the cycle length for the respective signal at a user interface. In a first example of the method, establishing the plurality of standardized ECG patterns includes continuously learning the waveform morphology of the real-time sensed ECG data from sensors monitoring heart activity and wherein the sensors include surface electrodes and intracardiac catheter electrodes. In a second example of the method, optionally including the first example, continuously learning the wave morphology of the real-time sensed ECG data includes filtering the real-time sensed ECG data through a band pass filter. In a third example of the method, optionally including one or both of the first and second examples, establishing the plurality of standardized ECG patterns includes using machine learning to establish standardized patterns based on deflections of the real-time sensed ECG data. In a fourth example of the method, optionally including one or more or each of the first through third examples, establishing the standardized patterns includes learning segments of the real-time sensed ECG data where the deflections occur and determining correlations among the learned segments. In a fifth example of the method, optionally including one or more or each of the first through fourth examples, selecting the signals of the real-time sensed ECG data includes, prior to establishing the plurality of standardized ECG patterns, placing a tick mark where a deflection of the deflections of the real-time sensed ECG data exceeds a first signal threshold. In a sixth example of the method, optionally including one or more or each of the first through fifth examples, selecting the signals of the real-time sensed ECG data includes identifying a first signal of the signals of the real-time sensed ECG data where a deflection exceeds a second signal threshold and has a correlation percentage with at least one of a target number of patterns of the plurality of standardized ECG patterns above a first correlation threshold, and wherein the target number of patterns is determined based on the learned segments of the real-time sensed ECG data where deflections occur prior to the first signal. In a seventh example of the method, optionally including one or more or each of the first through sixth examples, the method further comprises, replacing a previously established pattern of the target number of patterns with a new pattern when the target number of patterns is exceeded and the previously established pattern has a least number of instances of correlation with the signals of the real-time sensed ECG data. In an eighth example of the method, optionally including one or more or each of the first through seventh examples, selecting the signals of the real-time sensed ECG data includes displaying tick marks on the real-time sensed ECG data at the user interface, with a tick mark at each signal of the signals of the real-time sensed ECG data. In a ninth example of the method, optionally including one or more or each of the first through eighth examples, determining the cycle length includes determining a distance between a first tick mark of the tick marks, corresponding to a first signal of the signals of the real-time ECG data, and a second tick mark of the tick marks, corresponding to a second signal of the signals of the real-time ECG data.

The disclosure also provides support for a method for analyzing an electrocardiogram (ECG) in real-time, comprising, responsive to capturing segments of ECG data for a target duration of time, identifying periods of interest in the segments where a signal exceeds a first signal threshold, the signal indicating a heart activity, and establishing the periods of interest as learning templates, responsive to generation of a first target number of learning templates in a set of learning templates, determining a correlation of a waveform morphology among the set of learning templates and generating a final template based on a first correlation threshold, responsive to generation of a target number of final templates in a set of final templates, using the set of final templates to identify a recurrent signal in real-time based on a second correlation threshold, displaying a tick mark at a user interface to indicate the recurrent signal, and displaying a cycle length of the recurrent signal at the user interface, where the cycle length is a distance between successive tick marks. In a first example of the method, the target duration of time for capturing the segments of the ECG data is two seconds. In a second example of the method, optionally including the first example, determining the correlation of the waveform morphology among the set of learning templates and generating the final template based on the first correlation threshold includes, when a waveform morphology of a first learning template of the set of learning templates and waveform morphologies of each of a second target number of learning templates correlate at a percentage above the first correlation threshold, identifying the first learning template as the final template, and wherein the second target number of learning templates is less than the first target number of learning templates. In a third example of the method, optionally including one or both of the first and second examples, the method further comprises, prior to establishing a first final template, placing a tick mark where the signal exceeds a second signal threshold. In a fourth example of the method, optionally including one or more or each of the first through third examples, the first signal threshold is a first percentage of a signal range between a minimum value and a maximum value and the second signal threshold is a second percentage of the signal range and wherein the second percentage is greater than the first percentage. In a fifth example of the method, optionally including one or more or each of the first through fourth examples, generation of the set of final templates is continuous and, upon generation of final templates beyond the target number of final templates, replacing a final template of the set of final templates having a least number of instances of correlation with recurrent signals with a newly generated final template.

The disclosure also provides support for a method for detecting cardiac arrhythmia in real-time, comprising identifying a candidate beat from an electrocardiogram signal via machine learning, the electrocardiogram signal detected by one or more of surface electrodes and intracardiac catheter electrodes, and upon determination that an amplitude of the candidate beat is above a first threshold, estimating a correlation between a waveform morphology of the candidate beat and the waveform morphologies of a set of final templates, the set of final templates generated based on identification of a recurrent waveform morphology of the electrocardiogram signal, to confirm if the candidate beat is an actual beat, and displaying a tick mark and an estimated cycle length of the candidate beat at a user interface when the candidate beat is confirmed as the actual beat. In a first example of the method, the first threshold is a first percentage of a signal amplitude defined by a minimum value and a maximum value of the electrocardiogram signal. In a second example of the method, optionally including the first example, estimating the correlation between the waveform morphology of the candidate beat and the waveform morphologies of the set of final templates includes performing a correlation calculation and confirming the candidate beat is the actual beat when a resulting correlation percentage is greater than a second threshold. In a third example of the method, optionally including one or both of the first and second examples, the recurrent waveform morphology is identified where correlation of a first signal of the electrocardiogram signal exceeds a third correlation threshold when compared with at least three signals of the electrocardiogram signal.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method for an electrophysiology study, comprising:
establishing a plurality of standardized electrocardiogram (ECG) patterns based on real-time sensed ECG data;
selecting signals of the real-time sensed ECG data based on a correlation between the real-time sensed ECG data and at least one pattern of the plurality of standardized ECG patterns;
determining a cycle length of each of the signals of the real-time ECG data; and
displaying the cycle length for the respective signal at a user interface.

2. The method of claim 1, wherein establishing the plurality of standardized ECG patterns includes continuously learning the waveform morphology of the real-time sensed ECG data from sensors monitoring heart activity and wherein the sensors include surface electrodes and intracardiac catheter electrodes.

3. The method of claim 2, wherein continuously learning the wave morphology of the real-time sensed ECG data includes filtering the real-time sensed ECG data through a band pass filter.

4. The method of claim 1, wherein establishing the plurality of standardized ECG patterns includes using machine learning to establish standardized patterns based on deflections of the real-time sensed ECG data.

5. The method of claim 4, wherein establishing the standardized patterns includes learning segments of the real-time sensed ECG data where the deflections occur and determining correlations among the learned segments.

6. The method of claim 5, where selecting the signals of the real-time sensed ECG data includes, prior to establishing the plurality of standardized ECG patterns, placing a tick mark where a deflection of the deflections of the real-time sensed ECG data crosses a first signal threshold.

7. The method of claim 1, wherein selecting the signals of the real-time sensed ECG data includes identifying a first signal of the signals of the real-time sensed ECG data where a deflection crosses a second signal threshold and has a correlation percentage with at least one of a target number of patterns of the plurality of standardized ECG patterns above a first correlation threshold, and wherein the target number of patterns is determined based on the learned segments of the real-time sensed ECG data where deflections occur prior to the first signal.

8. The method of claim 7, further comprising, replacing a previously established pattern of the target number of patterns with a new pattern when the target number of patterns is exceeded and the previously established pattern has a least number of instances of correlation with the signals of the real-time sensed ECG data.

9. The method of claim 1, wherein selecting the signals of the real-time sensed ECG data includes displaying tick marks on the real-time sensed ECG data at the user interface, with a tick mark at each signal of the signals of the real-time sensed ECG data.

10. The method of claim 9, wherein determining the cycle length includes determining a distance between a first tick mark of the tick marks, corresponding to a first signal of the signals of the real-time ECG data, and a second tick mark of the tick marks, corresponding to a second signal of the signals of the real-time ECG data.

11. A method for analyzing an electrocardiogram (ECG) in real-time, comprising:
responsive to capturing segments of ECG data for a target duration of time;
identifying periods of interest in the segments where a signal crosses a first signal threshold, the signal indicating a heart activity, and establishing the periods of interest as learning templates;
responsive to generation of a first target number of learning templates in a set of learning templates;
determining a correlation of a waveform morphology among the set of learning templates and generating a final template based on a first correlation threshold;
responsive to generation of a target number of final templates in a set of final templates;
using the set of final templates to identify a recurrent signal in real-time based on a second correlation threshold;
displaying a tick mark at a user interface to indicate the recurrent signal; and
displaying a cycle length of the recurrent signal at the user interface, where the cycle length is a distance between successive tick marks.

12. The method of claim 11, wherein the target duration of time for capturing the segments of the ECG data is two seconds.

13. The method of claim 11, wherein determining the correlation of the waveform morphology among the set of learning templates and generating the final template based on the first correlation threshold includes, when a waveform morphology of a first learning template of the set of learning templates and waveform morphologies of each of a second target number of learning templates correlate at a percentage above the first correlation threshold, identifying the first learning template as the final template, and wherein the second target number of learning templates is less than the first target number of learning templates.

14. The method of claim 11, further comprising, prior to establishing a first final template, placing a tick mark where the signal crosses a second signal threshold.

15. The method of claim 14, wherein the first signal threshold is a first percentage of a signal range between a minimum value and a maximum value and the second signal threshold is a second percentage of the signal range and wherein the second percentage is greater than the first percentage.

16. The method of claim 11, wherein generation of the set of final templates is continuous and, upon generation of final templates beyond the target number of final templates, replacing a final template of the set of final templates having a least number of instances of correlation with recurrent signals with a newly generated final template.

17. A method for detecting cardiac arrhythmia in real-time, comprising:
identifying a candidate beat from an electrocardiogram signal via machine learning, the electrocardiogram signal detected by one or more of surface electrodes and intracardiac catheter electrodes; and
upon determination that an amplitude of the candidate beat crosses a first threshold;
estimating a correlation between a waveform morphology of the candidate beat and the waveform morphologies of a set of final templates, the set of final templates generated based on identification of a recurrent waveform morphology of the electrocardiogram signal, to confirm if the candidate beat is an actual beat; and
displaying a tick mark and an estimated cycle length of the candidate beat at a user interface when the candidate beat is confirmed as the actual beat.

18. The method of claim 17, wherein the first threshold is a first percentage of a signal amplitude defined by a minimum value and a maximum value of the electrocardiogram signal.

19. The method of claim 17, wherein estimating the correlation between the waveform morphology of the candidate beat and the waveform morphologies of the set of final templates includes performing a correlation calculation and confirming the candidate beat is the actual beat when a resulting correlation percentage is greater than a second threshold.

20. The method of claim 17, wherein the recurrent waveform morphology is identified where correlation of a first signal of the electrocardiogram signal exceeds a third correlation threshold when compared with at least three signals of the electrocardiogram signal.
